(19) United States
(12) Patent Application Publication
(10) Pub. No.: US 2024/0058095 A1
(43) Pub. Date: Feb. 22, 2024

(54) SOFT TISSUE MARKER FOR ATTACHMENT TO A SURGICAL EXCISION BED AND METHOD OF MARKING SURGICAL SITE

(71) Applicant: Merit Medical Systems, Inc., South Jordan, UT (US)

(72) Inventor: Ray Bricault, West Boylston, MA (US)

(21) Appl. No.: 18/451,523

(22) Filed: Aug. 17, 2023

Related U.S. Application Data
(60) Provisional application No. 63/399,316, filed on Aug. 19, 2022.

(51) Int. Cl. A61B 90/00

(52) U.S. Cl. CPC A61B 90/39; A61B 2090/3966; A61B 2090/3991; A61B 2090/3987

(57) ABSTRACT

In accordance with aspects of the inventive concepts, provided is a marker device, comprising: an elongate element; a needle disposed at a distal end of the elongate element; and at least one radiopaque marker element coupled to or disposed on at least a portion of an outer surface of the elongate element. The at least one marker can include radiopaque portions. The at least one radiopaque marker element can be or include a helical coil wrapped around a suture element. Also provided is a method of marking a surgical site comprising suturing a marker device into the surgical site. The method can include sewing or otherwise attaching the marker device to the soft tissue at boundaries of an excision site. Also provided is a method of making a marker device.

### RELATED CASES

This application claims priority to U.S. Provisional Application No. 63/399,316, filed on Aug. 19, 2022 and titled “SOFT TISSUE MARKER FOR ATTACHMENT TO A SURGICAL EXCISION BED AND METHOD OF MARKING SURGICAL SITE,” which is hereby incorporated by reference in its entirety.

### TECHNICAL FIELD

The present disclosure relates generally to the field of surgical markers at surgical sites. More particularly, some embodiments relate to apparatuses and methods for implanting and/or delivering markers to an excision site.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a marker device with a radiopaque marker element for soft tissue according to one embodiment of the present disclosure.

FIG. 2A illustrates a marker device with a radiopaque marker element for soft tissue according to one embodiment of the present disclosure.

FIG. 2B illustrates a detail view of the marker device of FIG. 2A.

FIG. 3A illustrates a schematic of a radiopacity pattern of a marker device formed by a radiopaque marker element according to one embodiment of the present disclosure.

FIG. 3B illustrates a schematic of a radiopacity pattern of a marker device formed by a radiopaque marker element according to one embodiment of the present disclosure.

FIG. 3C illustrates a schematic of a radiopacity pattern of a marker device formed by a radiopaque marker element according to one embodiment of the present disclosure.

### DETAILED DESCRIPTION

Various aspects of various embodiments of the present disclosure will be described more fully hereinafter with reference to the accompanying drawings, in which some exemplary embodiments are shown. The present embodiment may, however, be embodied in many different forms and should not be construed as limited to the exemplary embodiments set forth herein.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present disclosure. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. The phrase “coupled to” is broad enough to refer to any suitable coupling or other form of interaction between two or more entities, including mechanical interaction. Thus, two components may be coupled to each other even though they are not in direct contact with each other. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). The phrases “attached to” or “attached directly to” refer to interaction between two or more entities which are in direct contact with each other and/or are separated from each other only by a fastener of any suitable variety (e.g., mounting hardware or an adhesive).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in the figures is turned over, elements described as “below” and/or “beneath” other elements or features would then be oriented “above” the other elements or features. The device may be otherwise oriented (e.g., rotated 90 degrees or at other orientations) and the spatially relative descriptors used herein interpreted accordingly.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Exemplary embodiments are described herein with reference to cross-sectional illustrations that are schematic illustrations of idealized exemplary embodiments (and intermediate structures). As such, variations from the shapes of the illustrations as a result, for example, of manufacturing techniques and/or tolerances, are to be expected. Thus, exemplary embodiments should not be construed as limited to the particular shapes of regions illustrated herein but are to include deviations in shapes that result, for example, from manufacturing.

Excision is the removal from the body use a scalpel, laser, or other cutting tool. A surgical excision is usually done to remove a lump or other suspicious growth. Some normal tissue around the lump is usually removed at the same time. The boundaries of the excision site are three-dimensional and can be relatively complex based on the lump or suspicious tissue removed. When the lump or suspicious tissue is removed, a cavity is form that usually collapses on itself after the tissue is remove. This collapse of the tissue on the excision site can further complicate the original boundaries of the excision site.

The present disclosure is directed to an elongate marker device, method for making same, and the method of using the same for intraoperative attachment to the surgical bed in a patient. The marker device may be sewn or otherwise attached to the soft tissue of the patient and can identify the boundaries of the excision site when visualized by a diagnostic method, such as, for example, mammography, ultrasound, computerized tomography (CT) scans, magnetic resonance imaging (MRI) scans, fluoroscopy scans, X-rays, and the like. The marker device includes at least one marker element that is radiopaque. Accordingly, a surgeon may use elongate marker device to identify the original boundaries of the excision site in a three-dimensional space.

The elongate marker device can be used for targeting radiotherapy or other surgical procedures, including guidance to easily locate the surgical bed in cases where follow up surgery for re-excision is required (for example, in case of a positive margin report finding after a lumpectomy procedure). In various embodiments, the elongate marker device may be used for labeling lumpectomy cavity margins. However, the elongate marker device is not so limited and the elongate marker device can be used with other surgical procedures as well.

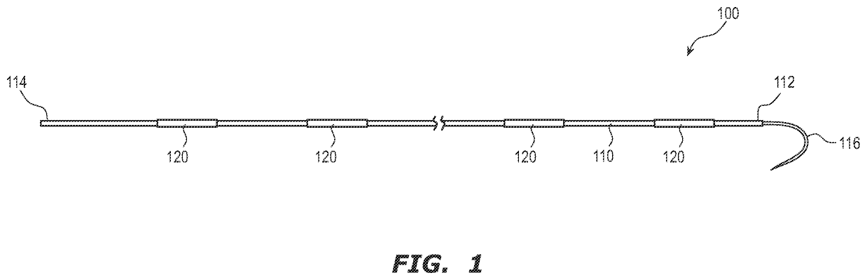

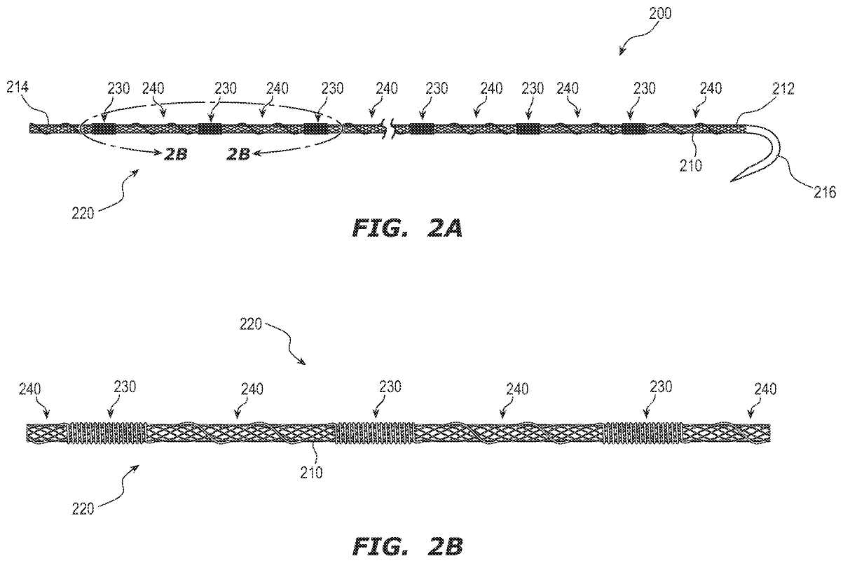

FIG. 1 illustrates a schematic of a marker device 100 according to one embodiment of the present disclosure. The marker device 100 comprises an elongate element 110 with a distal end 112, and a proximal end 114. The elongate element 110 may be an absorbable or non-absorbable (permanent) surgical suture. Exemplary materials of the elongate element 110 may include plain gut, chromic gut, fast gut, absorbable polymeric materials, poliglecaprone, polyglycolic acid, polyglactin and related polymers, polglyconate, polydioxanone closures, silk, nylon (monofilament and polyfilaments), braided polyester, polybutester, surgical steel, and the like. The elongate element 110 may be a monofilament or a polyfilament. FIG. 1 illustrates the elongate element 110 as a monofilament, whereas, FIGS. 2A and 2B illustrate the elongate element 210 as a polyfilament. In some embodiments, the elongate element 110 may be a polyfilament with fibers that are twisted or braided. A diameter of the elongate element 110 may range between 0.01 and 1.0 mm. In some embodiments, the size (gauge) of the elongate element 110 may range from USP 4-0 through USP 2. In some embodiments, the elongate element 110 may be a fine, biocompatible, radiopaque thread or wire.

In some embodiments, the distal end 112 of the elongate element 110 may comprise a needle 116. The needle 116 may be a surgical needle and may be coupled to the distal end 112 of the elongate element 110 to facilitate attachment to, or insertion into, of the elongate element 110 into soft tissue. In other words, the needle 116 may promote tissue access/attachment enabling a surgeon to suture the marker device 100 into place at the surgical site. The needle 116 may have a variety of different shapes, such as straight, ¼ circle, ⅜ circle, ½ circle, ⅝ circle, compound curved, half curved, half curved at both ends of a straight segment, and the like. A point of the needle 116 may have a variety of different geometries, such as taper, cutting, reverse cutting, trocar point or tapercut, blunt points, side cutting or spatula points, atraumatic, and the like. The needle 116 maybe a combination of any one of the above noted needle shapes and the above noted needle tip geometries.

The marker device 100 may further include at least one radiopaque marker element 120 that is attached to or disposed on or around an outer surface of the elongate element 110. The radiopaque marker element 120 may be visible under mammography, ultrasound, computerized tomography (CT) scans, magnetic resonance imaging (MRI) scans, fluoroscopy scans, X-rays, and the like. In some embodiments, the marker device 100 is configured to enhance or minimize the visibility of a given design of a marker device 100 via one of the different imaging modalities disclosed above.

In the illustrated embodiment of FIG. 1, the marker device 100 comprises one or more radiopaque marker elements 120 that are spaced apart from one another along the length of the elongate element 110. In some embodiments, the one or more radiopaque marker elements 120 may be evenly spaced apart from each other. In some embodiments, the one or more radiopaque marker elements 120 may be unevenly spaced apart from each other. In some embodiments, the one or more radiopaque marker elements 120 may be connected together by a wire. In some embodiments, the one or more radiopaque marker elements 120 may be unconnected. In some embodiments, the one or more radiopaque marker elements 120 may be positioned along a portion of the elongate element 110 in a predetermined pattern. In some embodiments, the pattern may be a repeating pattern. In some embodiment, the one or more radiopaque marker elements 120 create a unique pattern along a portion of the elongate element 110.

The one or more radiopaque marker elements 120 may have a variety of different shapes or structures. For example, in one embodiment, the one or more radiopaque marker elements 120 may have a tubular shape that is disposed around the elongate element 110. In another embodiment, the one or more radiopaque marker elements 120 may take the form of a helical coil wrapped around the outer surface of the elongate element 110. In another embodiment, the one or more radiopaque marker elements 120 may be coupled to the elongate element 110. In another embodiment, the one or more radiopaque marker elements 120 may be stamped onto the elongate element 110. In some embodiments, the one or more radiopaque marker elements 120 may be integral with the elongate element 110. In another embodiment, the radiopaque marker elements 120 may be inserted into a braided elongate element 110. In another embodiment, the one or more radiopaque marker elements 120 may be combination of the above noted embodiments.

FIGS. 2A and 2B illustrate an embodiment of a marker device 200 that resembles the marker device 100 described above in certain respects. Accordingly, like features are designated with like reference numerals, with the leading digits incremented to “2.” For example, the embodiment depicted in FIG. 2 includes a elongate element 210 that may, in some respects, resemble the elongate element 110 of FIG. 1. Relevant disclosure set forth above regarding similarly identified features thus may not be repeated hereafter. Moreover, specific features of the marker device 100 and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the marker device 200 and related components depicted in FIGS. 2A and 2B. Any suitable combination of the features, and variations of the same, described with respect to the marker device 100 and related components illustrated in FIG. 1 can be employed with the marker device 200 and related components of FIG. 2, and vice versa. This pattern of disclosure applies equally to further embodiments depicted in subsequent figures and described hereafter, wherein the leading digits may be further incremented.

FIG. 2A illustrates the marker device 200 according to one embodiment of the present disclosure. The marker device 200 includes the elongate element 210 with a distal end 212 and a proximal end 214. A needle 216 may be disposed at the distal end 212 of the elongate element 210. In some embodiment, the needle 216 is coupled to the distal end 212 of the elongate element 210. The marker device 200 further includes a radiopaque marker element 220 that is disposed along a length of the elongate element 210.

In the illustrated embodiment, the radiopaque marker element 220 of the marker device 200 is a helical coil that wraps around an outer surface of the elongate element 210. The helical coil may be a platinum-iridium or another suitable radiopaque material.

The radiopaque marker element 220 may comprise a pattern of coils or sets of patterned coils along the length of the marker device 200. While the radiopaque marker element 220 is illustrated as extending along an entire length of the elongate element 210, in some embodiments, the radiopaque marker element 220 may only extend a portion of the elongate element 210. The coil pattern of the radiopaque marker element 220 may include different winding densities, providing various levels of radiopacity. The marker device 200 may be employed to mark multiple locations in an excision site and the different winding densities provide different levels of radiopacity may help the surgeon correctly identify locations in the excision bed based on the different levels of radiopacity. For example, the radiopaque marker element 220 may mark the boundaries of the excision site in a three-dimensional space.

FIG. 2A illustrates the radiopaque marker element 220 comprising a plurality of high-density portions 230 separated by a plurality of low-density portions 240. FIG. 2B illustrates a detailed schematic of the marker device 200 illustrating three high-density portions 230 which are separated by low-density portions 240. In some embodiments, the radiopaque marker element 220 is a continuous coil that extends along a portion of the elongate element 210. In some embodiments, the high-density portions 230 and the low-density portions are unconnected.

FIGS. 2A and 2B illustrate an exemplary pattern of the high-density portions 230 relative to the low-density portions 240 of the radiopaque marker element 220. The radiopaque marker element 220 may be tightly wrapped (e.g., high-density) which may optimize the tactile and visual feedback to the surgeon or can be loosely wrapped (e.g., low-density) to minimize tactile and visual feedback and allow the marker device 200 to be more flexible or any combination thereof. In some embodiments, the tightly wrapped radiopaque marker element 220 may be fully dense, such as embodiments wherein adjacent coils or wraps are in contact with each other. The high-density portions 230 may have different densities that range from 100% (wherein each revolution of the coil is in direct contact with the adjacent revolution) to 25%. The 25% density is determined based of the 100% density. For example, a portion with 100% density may be understood as a portion where adjacent coils are in contact with each other and 100% of the surface area of a given length of the elongate element 210 is covered with coils. Similarly, a portion with 25% density may be understood as a portion wherein 25% of the surface area of a given length of the elongate element 210 is covered with coils. Furthermore, in some embodiments, the density may be described in the number of coils per unit length.

In some embodiments, the radiopaque marker element 220 can be attached to the elongate element 210 in addition to being wrapped around the outer surface of the elongate element 210. For example, the helical coil of the radiopaque marker element 220 may be attached to the elongate element 210 by extending a wire of the helical coil through the elongate element 210, partially extending a wire through the elongate element 210 or the braid of the elongate element 210 at a predetermined point or a plurality of predetermined points along the length of the elongate element 210.

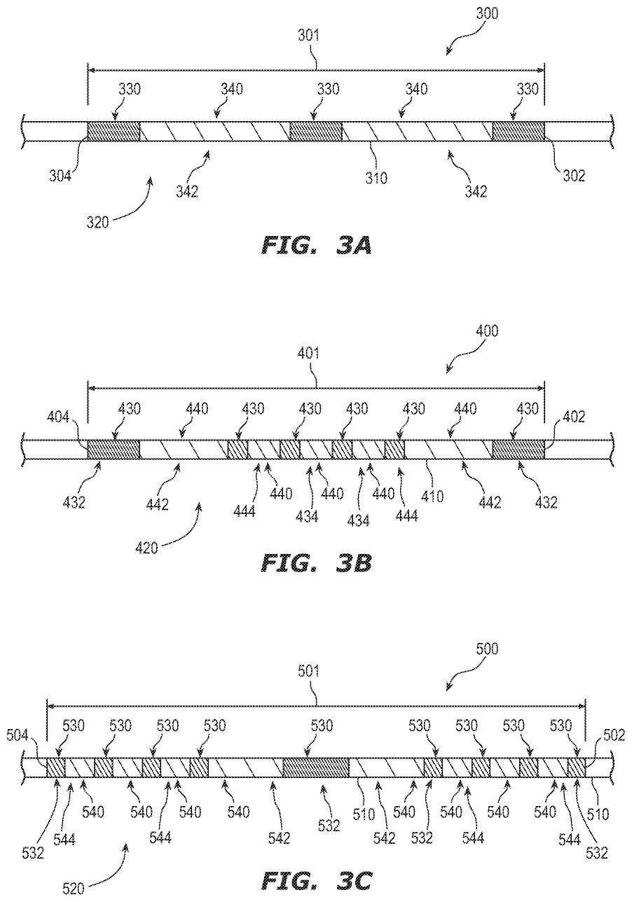

FIGS. 3A-3C illustrate a variety of different embodiments of patterns of radiopacity of a marker device formed by a radiopaque marker element. In various embodiments, the coil patterns of the radiopaque marker element are useful not only for marking the location of the excision site in soft tissue, but also in defining the orientation of the excision site, such as anterior vs. posterior and/or medial vs. lateral, based on the patterns of radiopacity of the radiopaque marker element.

The pattern of radiopacity of the marker device can also be used to either enhance or diminish visibility by the naked eye and/or creating tactile (in form of a differential in friction on insertion or rigidity) feedback for the surgeon when the marker device is attached. The pattern of radiopacity may include high-density portions and low-density portions.

The pattern of radiopacity of the marker device can be repeated along the length of the marker device so as to facilitate the surgeon sewing one marker device in place, cutting the remaining length of the elongate element of the marker device and repeating the process with another marker device. Further, the individual coiled marker sets may be varied along the length of the elongate element specifically in correlation to the region of tissue being marked to provide information on orientation, such as anterior-posterior, medial-lateral, and/or other orientation or position details that can be seen on imaging. The length of the bands or banding patterns may range from 0.5 to 28 cm.

FIG. 3A illustrates a schematic of a portion 301 of a marker device 300 with a distal end 302 and a proximal end 304 according to one embodiment of the present disclosure. The marker device 300 includes an elongate element 310 and a radiopaque marker element 320 that is disposed along a length of the elongate element 310. While not illustrated, the marker device 300 may include a needle at a distal end of the elongate element 310. The radiopaque marker element 320 is a helical coil that is wrapped around an outer surface of the elongate element 310. While the illustrated embodiment of FIG. 3A illustrates a single radiopaque marker element 320, the marker device 300 may include more than one radiopaque marker element 320.

The radiopaque marker element 320 comprises a plurality of high-density winding portions 330 and a plurality of low-density winding portions 340 to form a predetermined pattern of radiopacity. The low-density winding portions 340 separate the high-density winding portions 330 from each other. In the illustrated embodiment of FIG. 3A, the pattern of radiopacity of the radiopaque marker element 320 includes three high-density winding portions 330 separated by low-density winding portions 340. One of the high-density winding portions 330 is disposed at the distal end 302 of the portion 301, one high-density winding portions 330 is disposed at the proximal end 304 of the portion 301, and the other high-density winding portion 330 is disposed between the proximal and distal high-density winding portions 330. The high-density winding portions 330 may be tightly wound so that the high-density winding portions 330 are higher density portions and/or fully dense portions. In some embodiments, the high-density winding portions are 100% dense. In other words, each revolution of the coil of the radiopaque marker element 320 is in direct contact with the adjacent revolution. The length of the high-density winding portions 330 may be about 3 mm, and low-density winding portions 340 may be about 6 mm for an overall length of the portion 301 being about 27 mm. The illustrated portion 301 of the elongate element 310 may be repeated over the length of the elongate element 310.

FIG. 3B illustrates a schematic of a portion 401 of a marker device 400 with a distal end 402 and a proximal end 404 according to one embodiment of the present disclosure. The marker device 400 includes an elongate element 410 and a radiopaque marker element 420 that is disposed along a length of the elongate element 410. While not illustrated, the marker device 400 may include a needle at a distal end of the elongate element 410. The radiopaque marker element 420 is a helical coil that is wrapped around an outer surface of the elongate element 410. While the illustrated embodiment of FIG. 3B illustrates a single radiopaque marker element 420, the marker device 400 may include more than one radiopaque marker element 420.

The radiopaque marker element 420 comprises a plurality of high-density winding portions 430 and a plurality of low-density winding portions 440 to form a predetermined pattern of radiopacity. The low-density winding portions 440 separate the high-density winding portions 430 from each other. In the illustrated embodiment of FIG. 3B, the pattern of radiopacity of the radiopaque marker element 420 may include high-density winding portions 430 with different radiopacities. The pattern of radiopacity may include two high-density winding portions 432 disposed at the distal end 402 of the portion 401 and the proximal end 404 of the portion 401 and four high-density winding portions 434 disposed between the distal end 402 and the proximal end 404, each of the high-density winding portions 432, 434 separated by low-density winding portions 440. The high-density winding portions 432 may be tightly wound so that the high-density winding portions 432 are higher density portions and/or fully dense portions. In some embodiments, the high-density winding portions are 100% dense. In other words, each revolution of the coil of the radiopaque marker element 420 is in direct contact with the adjacent revolution. The length of the high-density winding portions 432 may be about 2.5 mm. The high-density winding portions 434 may be wound so that the high-density winding portions 434 are about 35% density relative to a fully dense portion. The length of the high-density winding portions 434 may be about 1 mm. The low-density winding portions 440 may comprise portions of different lengths. A pattern of radiopacity may include low-density winding portions 442 of lengths of about 5 mm and low-density winding portions 444 of lengths of about 2 mm. The overall length of the portion 401 may be about 24 mm. The illustrated portion 401 of the elongate element 410 may be repeated over the length of the elongate element 410.

FIG. 3C illustrates a schematic of a portion 501 of a marker device 500 with a distal end 502 and a proximal end 504 according to one embodiment of the present disclosure. The marker device 500 includes an elongate element 510 and a radiopaque marker element 520 that is disposed along a length of the elongate element 510. While not illustrated, the marker device 500 may include a needle at a distal end of the elongate element 510. The radiopaque marker element 520 is a helical coil that is wrapped around an outer surface of the elongate element 510. While the illustrated embodiment of FIG. 3C illustrates a single radiopaque marker element 520, the marker device 500 may include more than one radiopaque marker element 520.

The radiopaque marker element 520 comprises a plurality of high-density winding portions 530 and a plurality of low-density winding portions 540 to form a predetermined pattern of radiopacity. The low-density winding portions 540 separate the high-density winding portions 530 from each other. In the illustrated embodiment of FIG. 3C, the pattern of radiopacity of the radiopaque marker element 520 may include high-density winding portions 530 with different radiopacities. The pattern of radiopacity may include a central high-density winding portion 532 and four high-density winding portions 534 disposed on both sides (e.g., proximal and distal) of the central high-density winding portion 532, each of the high-density winding portions 532, 534 separated by low-density winding portions 540. The high-density winding portion 532 may be tightly wound so that the high-density winding portion 532 is a higher density portion and/or a fully dense portion. In some embodiments, the high-density winding portions are 100% dense. In other words, each revolution of the coil of the radiopaque marker element 420 is in direct contact with the adjacent revolution. The length of the high-density winding portions 532 may be about 5 mm. The high-density winding portions 534 may be wound so that the high-density winding portions 534 are about 35% density relative to the fully dense portion. The length of the high-density winding portions 534 may be about 1 mm. The low-density winding portions 540 may comprise portions of different lengths. A pattern of radiopacity may include low-density winding portions 542 of lengths of about 5 mm and low-density winding portions 544 of lengths of about 2 mm. The overall length of the portion 501 may be about 35 mm. The illustrated portion 501 of the elongate element 510 may be repeated over the length of the elongate element 510.

The present disclosure discloses the above noted patterns of radiopacity, however, other patterns of radiopacity are within the scope of this disclosure.

In some embodiments, a marker device may comprise a radiopaque marker element that is nonrepeating but is unique along a length of a portion the elongate member. In some embodiments, that we be the entire length of the elongate member. In some embodiment, the length of the portion may be less than the entire length of the elongate element. In some embodiments, the length of the portion may be half of the elongate element.

In some embodiments, the elongate member may comprise more than one unique pattern of radiopacity. For example, in one embodiment, a proximal portion of the elongate element may have a unique pattern of radiopacity, a central portion of the elongate element may have a unique pattern of radiopacity, and a distal portion of the elongate element may have a unique pattern of radiopacity, each pattern being different from the other patterns of radiopacity. Accordingly, a surgeon may use the same marker device to make at least three unique identifying patterns of radiopacity at the excision site to mark specific boundaries or locations. For example, an upper boundary, a center, and a lower boundary. However, the present disclosure is not limited to three unique patterns but the marker device may have more or less than three unique patterns of radiopacity along the elongate element.

Returning to FIG. 2, the marker device 200 may be manufactured in a number of ways. In some embodiments, a first step includes providing an elongate element 210, such as a suture. The suture may be a wire or a thread (e.g., monofilament or a polyfilament). A radiopaque marker element 220 may be coupled to the elongate element 210. Coupling the radiopaque marker element 220 to the elongate element 210 includes wrapping a radiopaque helical coil around an outer surface of the elongate element 210. In some embodiments, the radiopaque marker element 220 may be inserted through the filaments of a polyfilament elongate member 210. In some embodiments, the radiopaque marker element 210 is intertwined with the braid of a polyfilament elongate member 210. The needle 216 may be coupled to the distal end 212 of the elongate element 210.

In some embodiments, the wrapping of the radiopaque helical coil around the outer surface of the elongate element 210 includes wrapping a plurality of high-density portions 230 around the outer surface of the elongate element 210 and wrapping a plurality of low-density portions 240 around the outer surface of the elongate element 210. As discussed above, the high-density portions 230 and the low-density portions 240 have different winding densities and thus have different radiopacities. The high-density portions 230 may have different densities that range from 100% (wherein each revolution of the coil is in direct contact with the adjacent revolution) to 25%. The 25% density is determined based of the 100% density.

The high-density portions 230 are separated from each other by the low-density portions 240. In some embodiments, the radiopaque helical coil is a continuous wire. In some embodiments, the marker device 200 may include a plurality of radiopaque helical coils. In some embodiments, there may be a plurality of radiopaque helical coils wrapped around the elongate element 210 that may be connected or unconnected from each other.

In some embodiments, the radiopaque helical coil may be stretched after the radiopaque helical coil is wrapped around the elongate element 210. For example, one or more portions of the helical coil may be stretch to change the winding density of a portion of the radiopaque helical coil to change the radiopacity of the portion of radiopaque helical coil. By stretching the radiopaque helical coil, the stretched portion of the radiopaque helical coil has a lower density and a lower radiopacity.

A pattern of radiopacity may be implemented on the marker device 200 with the one or radiopaque marker element 220. The pattern of radiopacity may be a repeating pattern, such as one of the patterns of radiopacity disclosed in relation to FIGS. 3A-3C. In some embodiments, the pattern of radiopacity may be unique along the entire length radiopacity of the marker device 200. In some embodiments, the pattern of radiopacity may extend the entire length of the elongate element 210. In some embodiments, the pattern of radiopacity may only extend a portion of the elongate element 210.

The marker device 200 may be used to suture a surgical site. As discussed, the marker device 200 includes the elongate element 210, which may be a suture, the needle 216 disposed at the distal end 212 of the elongate element 210, and the radiopaque marker element 220. The marker device 200 may include one or more radiopaque marker elements 220. The radiopaque marker element 220 may be helically coiled around at least a portion of an outer surface of the elongate element 210. The radiopaque marker element 220 may include a plurality of different winding densities with each wining density providing a different radiopacity.

A surgeon may sew soft tissue together with marker device 200 at the boundaries of an excision site. The radiopaque marker element 220 on the marker device 200 may be used to mark the excision site that will help with the identification of the excision site. For example, the radiopaque marker element may help a surgeon identify the boundaries of the excision site if the surgeon needs to reaccess the excision site in a different surgery after the after a first excision surgery is over.

After the surgeon has sutured the predetermined location with the marker device 200 leaving a pattern of radiopacity based on the radiopaque marker element 220, the surgeon may remove the needle 216 from the marker device 200. In most situations, the surgeon with remove the needle 216 and any of the remaining elongate element 210 not needed for suture. In some embodiments, the surgeon may use the remaining elongate element 210 and the needle 216 to make another suture by sewing soft tissue together at the excision site.

As discussed above, in some embodiments, the marker device may comprise a plurality of unique patterns of radiopacity on a single marker device. The surgeon may use the proximal portion of the elongate element of the marker device to make a first suture with a first pattern of radiopacity. Once the suture is completed, the surgeon may cut off the needle and the remaining elongate element of the marker device and the surgeon may use a central portion of the elongate element to make a second suture with a second unique pattern of radiopacity. This may be repeated for a distal portion of the elongate element to make a third suture with a third unique pattern of radiopacity. This may be repeated for the number of unique patterns of radiopacity on the marker device.

While the foregoing has described what are considered to be the best mode and/or other preferred embodiments, it is understood that various modifications can be made therein and that the invention or inventions may be implemented in various forms and embodiments, and that they may be applied in numerous applications, only some of which have been described herein. It is intended by the following claims to claim that which is literally described and all equivalents thereto, including all modifications and variations that fall within the scope of each claim.

It is appreciated that certain features of the invention, which are, for clarity, described in the context of separate embodiments, may also be provide in combination in a single embodiment. Conversely, various features of the invention which are, for brevity, described in the context of a single embodiment may also be provided separately or in any suitable sub-combination.

For example, it will be appreciated that all of the features set out in any of the claims (whether independent or dependent) can combined in any given way.

Recitation in the claims of the term “first” with respect to a feature or element does not necessarily imply the existence of a second or additional such feature or element. It will be apparent to those having skill in the art, with the benefit of this disclosure, that changes may be made to the details of the above-described embodiments without departing from the underlying principles of the present disclosure.

1. A marker device, comprising:
an elongate element;
a needle disposed at a distal end of the elongate element; and
at least one radiopaque marker element coupled to or disposed on at least a portion of an outer surface of the elongate element.

2. The device of claim 1, wherein the elongate element is suture.

3. The device of claim 1, wherein the at least one radiopaque marker includes portions having different radiopacities.

4. The device of claim 1, wherein the at least one radiopaque marker element includes a helical coil.

5. The device of claim 4, wherein the helical coil is wrapped around the elongate element.

6. The device of claim 5, wherein the helical coil includes portions having different winding densities, and wherein the different winding densities have different radiopacities.

7. The device of claim 5, wherein the helical coil includes a plurality of high-density winding portions that are spaced apart along at least a portion of the elongate element.

8. The device of claim 7, wherein the plurality of high-density winding portions are unconnected.

9. The device of claim 5, wherein the helical coil includes a plurality of high-density winding portions that are separated by low-density winding portions along at least a portion of the elongate element.

10. The device of claim 9, wherein the plurality of high-density winding portions comprise different radiopacities.

11. The device of claim 9, wherein the high-density winding portions and the low-density winding portion create a pattern of radiopacity.

12. The device of claim 11, wherein the pattern of radiopacity repeats along the elongate element.

13. The device of claim 4, wherein the at least one radiopaque marker element is configured for varying tactile and visual feedback via modifications of a coil density of the helical coil.

14. The device of claim 1, wherein the at least one radiopaque marker element includes a plurality of connected radiopaque marker elements.

15. A method of manufacturing a marker device, comprising:
providing an elongate element; and
wrapping a radiopaque helical coil around the elongate element.

16. The method of manufacturing of claim 15, further comprising coupling a needle to a distal end of the elongate element.

17. The method of manufacturing of claim 15, wherein wrapping the radiopaque helical coil includes wrapping a plurality of high-density winding portions and wrapping a plurality of low-density winding portions, wherein the high-density winding portions are separated by the plurality of low-density winding portions.

18. The method of manufacturing of claim 15, further comprising stretching one or more portions of the helical coil to form a radiopaque marker element having different winding densities, wherein the different winding densities have different radiopacities.

19. The method of manufacturing of claim 15, wherein the elongate element is suture.

20. A method of suturing a marker device comprising
obtaining a marker device comprising:
an suture;
a needle disposed at a distal end of the suture; and
a radiopaque marker element helically coiled around on at least a portion of an outer surface of the suture, wherein the radiopaque marker comprises a plurality of different winding densities, each winding density providing a different radiopacity;
suturing the marker device at a surgical site by sewing soft tissue at boundaries of an excision site, the different winding densities of the radiopaque marker providing different radiopaque levels for identifying locations of the excision site based on the different radiopaque levels; and
removing the needle from the marker device.
